(19) United States
(12) Patent Application Publication
(10) Pub. No.: US 2022/0022805 A1
(43) Pub. Date: Jan. 27, 2022

(54) SEIZURE DETECTION VIA ELECTROOCULOGRAPHY (EOG)

(71) Applicant: Eysz Inc., Piedmont, CA (US)

(72) Inventors: Rachel Kuperman, Piedmont, CA (US); Bikramjit Sarkar, San Diego, CA (US); Parth Amin, Cary, NC (US)

(21) Appl. No.: 17/381,562

(22) Filed: Jul. 21, 2021

Related U.S. Application Data
(60) Provisional application No. 63/055,075, filed on Jul. 22, 2020.

(51) Int. Cl. A61B 5/00; A61B 5/16; A61B 5/369; A61B 5/398

(52) U.S. Cl. CPC A61B 5/4094; A61B 5/163; A61B 5/746; A61B 5/398; A61B 5/7267; A61B 5/369; A61B 5/4836

(57) ABSTRACT

This invention incorporates analysis of electrooculographic (EOG) data into a methods and apparatus for reporting, alarming and intervening events and conditions. More particularly, EOG signals are analyzed separately from EEG signals, with the EOG signals used as a distinct source of information. The approach can identify patterns associated or predictive of seizures, syncope, drowsiness and loss of consciousness during night or day through the analysis of eye-movements recorded using just the EOG.

This patent application claims priority to a co-pending U.S. Provisional Patent Application Ser. No. 63/055,075 filed Jul. 22, 2020 entitled “Seizure Detection via Electrooculography (EOG), the entire contents of which are hereby incorporated by reference.

### BACKGROUND

This patent application relates to devices and methods for identifying patterns associated or predictive of seizures, syncope, drowsiness, loss of consciousness, or other neurological events or conditions through the analysis of eye-movements recorded using electrooculography (EOG).

An electroencephalogram (EEG) is a test used to evaluate electrical activity in the brain. An EEG tracks and records brain wave patterns. In the typical approach, small flat metal discs called electrodes are attached to the scalp with wires. The electrodes analyze the electrical impulses in the brain and send signals to a computer that records the results. Trained medical personnel can review the signals to assess whether there are abnormal patterns indicative of seizures or other brain disorders.

EEG-based monitoring is time-consuming, however, and requires experts to interpret EEG signals to detect seizures in patients. As such, automated methods have been devised to interpret EEGs. One approach uses a method called adaptive slope of wavelet coefficients counts over various thresholds (ASCOT) to classify patient episodes as seizure waveforms. See Lee, M. et al. “Automated epileptic seizure waveform detection method based on the feature of the mean slope of wavelet coefficient counts using a hidden Markov model and EEG signals”, ETRI Journal, 2020; 42(2):217-229.

International Patent Publication WO2019/173106A1 titled “Method of Detecting and/or Predicting Seizures” (filed by the Children's Hospital & Research Center of Oakland) (incorporated by reference herein) describes various methods for detecting and/or predicting an epileptic event in a subject with or without performing an EEG. This patent focuses on using eye movements recorded by video to identify seizures or loss of consciousness. Video based eye tracking is non-contact and does not require electrodes. It additionally can measure pupil size, gaze position, blink and other open eye movements and is incorporated into multiple devices.

Detection of, and alarming for epileptic seizures via non-invasive, non-EEG (electro-encephalography) body signals has also been published elsewhere, such as in Van de Vela, et al. “Non-EEG seizure detection systems and potential SUDEP prevention: State of the art review and update”, Seizure 41 (2016) 141-153.

Electrooculography (EOG) is another existing method to record relative eye-movements using electrical signals from electrodes placed on the skin near the eyes. EOG data is generated from the muscles around the eye and the dipole produced by the difference between the cornea and the retina. When EOG data is produced as part of an EEG, the EOG data has typically been thought of as an artifact when produced in conjunction with an EEG. In particular, the EOG data is subtracted out so that the electrical activity of the brain can be analyzed for seizure activity. Examples are described in Coelho, et al., “Electro-oculogram and submandibular montage to distinguish different eye, eyelid, and tongue movements in electroencephalographic studies”, Clin Neurophysiol., 2018 November; 129(11):2380-2391.

EOG as compared to video based eye tracking requires skin contact with an electrode, and does not require a camera. EOG can be collected as part of EEG. EOG can detect extraocular muscle activation which may or may not be detectable by video. Unlike video based eye tracking EOG can be used to identify eye movements when the eyes are closed. EOG can be used to identify relative eye position, and blink but not exact eye position or pupil size.

### BRIEF SUMMARY

This invention incorporates analysis of EOG data into a method of reporting, alarming and intervening. More particularly, our invention analyses EOG signals separately from EEG signals, with the EOG signals used as a distinct source of information that is complementary to the EEG. The approach can identify patterns associated with or predictive of seizures, syncope, drowsiness and loss of consciousness during night or day through the analysis of eye-movements recorded using just EOG.

The approach is to measure relative eye movement through EOG and then analyze such measurements via one or more algorithms to produce unique information about clinical state of consciousness, independent of the EEG. Although EEG can identify states of wakefulness, drowsiness and sleep, EEG cannot differentiate if a person has lost consciousness from a seizure or if epileptiform activity is associated with a change in responsiveness/consciousness. EEG may show seizure activity and EOG may show that normal eye movement is lost, associated with clinical change. Thus, EEG is a biomarker of the brain's electrical activity whereas EOG is a biomarker for consciousness impairment or the clinical/functional outcome of the electrical activity.

The EOG signals are converted to digital form, and the resulting EOG data can then be transmitted to a computing device for storage and further analysis. In one example approach, the EOG data is converted into relative eye-movement vectors to analyze the resulting change in eye-movement.

Subsequent automated analysis may involve using data processors to apply various signal processing, pattern recognition, artificial intelligence, neural networks, machine learning, and/or other techniques to detect seizures, syncopes, drowsiness, loss of consciousness, or other neurological events or conditions. Once analyzed, information (e.g. reports, alerts) may be sent electronically to another computing device for further post processing (e.g., to make a medical decision).

### BRIEF DESCRIPTION OF THE DRAWINGS

FIG. 1 is a high level diagram of a system and/or method and/or device for identifying patterns associated or predictive of seizures, syncope, drowsiness, loss of consciousness, or other neurological events or conditions through the analysis of eye-movements recorded using electrooculography (EOG).

FIG. 2 is a more detailed view of data flow, including pre-processing steps (signal processing methods), prior to ingestion by seizure detection.

FIG. 3 is a schematic diagram of the development methodology, which utilizes Physician interpretation to produce expert labels that serve as ground-truth for seizure detection training.

FIG. 4 is a chart comparing the approach described herein with other non-invasive methods such as video-based eye-tracking.

### DETAILED DESCRIPTION OF A PREFERRED EMBODIMENT

Electrooculography (EOG) is a method of measuring the electrical activity of the eye derived from the corneo-retinal standing potential that exists between the front and the back of the human eye, the extraocular muscles and eyelid through one or more electrodes placed near the eye.

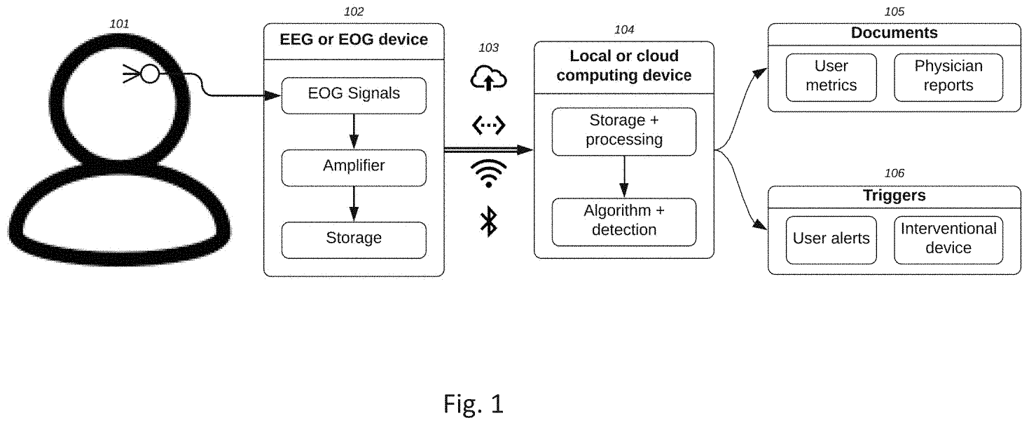

With reference to FIG. 1, EOG signals representing relative eye movement of an individual subject [101] can be obtained from a variety of methods or devices such as a mobile device, a wearable, or stationary EEG systems and/or EOG specific systems [102]. The electrodes may be incorporated into a head-worn eye-glass form factor, or other wearable device, or separate electrodes placed around the eye of the subject [101]. An example of a wearable form factor is the Jins MEME device, with a triple-electrode sensor placed between the eyes above the nose bridge. This configuration allows signal differencing to emphasize the EOG component of the electrical recordings and suppress other artifacts, such as direct cranial activity. Similar data can be obtained with standard EEG equipment, by choosing multiple electrode placements manually around the eyes, as may be customarily done for EOG research studies.

The methods and/or devices produce one or more EOG signals that may be sent to an amplifier and converted to digital form to provide EOG data [103]. The EOG data may be optionally stored at this point, before being communicated to a computing device [104]. Any suitable communication connection or network may be used such as Bluetooth, wired or wireless local area network, cellular data networks and the like [103]. Although a separate computing device [104] is shown here, it should be understood that the components shown in FIG. 1 may all be integrated into a single device or distributed among several electronic and/or computing devices.

The computing device may then further continuously record and store the EOG data, such as for current and/or later processing (analysis) [10], which may be done locally (such as for real-time processing), or remotely in the cloud (such as to generate offline reports at a later time).

In some implementations, the EOG signals and/or EOG data may be derived from a device that produces other streams of data such as an EEG device, or the EOG data may be combined with data from other types of systems, such as video-based eye tracking systems, gyroscopic systems, or other types of eye-tracking systems. It should therefore be understood that the EOG data [103] as mentioned herein may optionally include other data produced by such other sources to provide further information indicative of relative eye-movement.

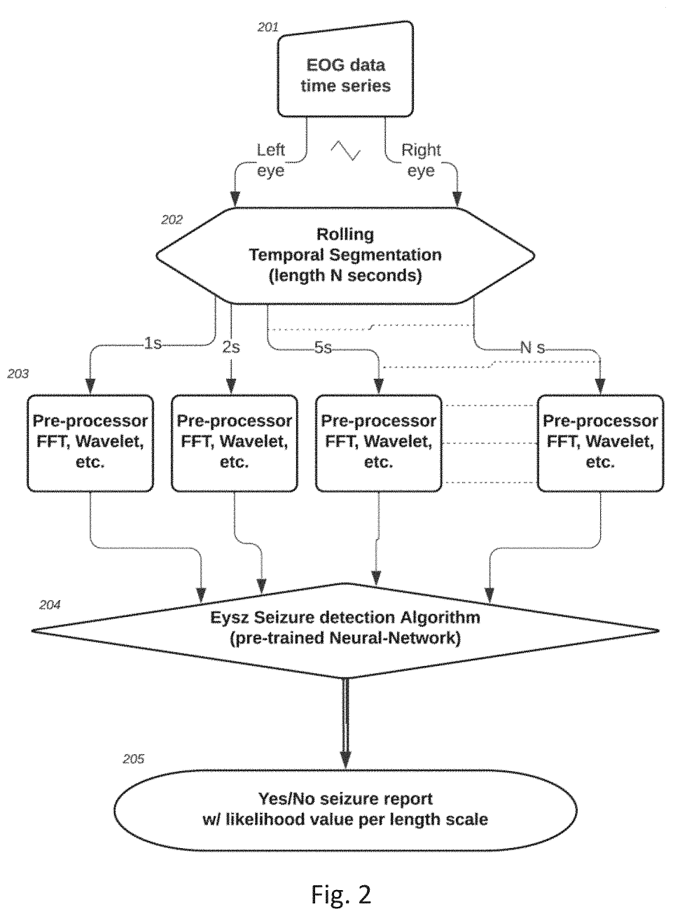

The EOG data [103] is then programmatically analyzed to identify and record patterns associated with, or predictive of, seizures, syncope, drowsiness, loss of consciousness, or other neurological events or conditions. The automated analysis may involve several steps, including converting the “electrical” activity (e.g., EOG data) into relative eye movement vectors; applying various algorithms and other available eye-related data to analyze the resulting change in movement; which then result in detecting loss of normal movement associated with seizure (as compared to previous detections); and further detecting (identifying) loss of consciousness or other conditions. The analysis and detecting steps are described in more detail in connection with FIG. 2.

Once the process of recording, analysis and identification have been completed, a combination of one or more outputs may then be generated, in some instances, as structured information. Some examples of these outputs include, but are not limited to:
1. Information is gathered and a structured digital document may be produced [105] that may include metrics or physician reports:
   A. For viewing on another computing device; and/or
   B. For transmission to a remote system for storage
2. A digital or electronic signal may be generated and digitally or electronically communicated to another device [106]:
   A. To generate an alarm, a user alert, notification, and/or electronic message on such other device(s), such as on a smartphone or hospital monitoring system; and/or.
   B. To communicate with an interventional or closed loop device, such as a Vagus Nerve Stimulator (VNS) or Responsive Neurostimulation System (RNS) which may enable a caregiver/physician or assistive device to then take action to mitigate the seizure.

FIG. 2 shows one approach to the types of signal processing and analysis that may be employed by the processing step 104. It should be understood that this is but one example and other approaches are possible.

In this example, EOG data is received as a time series (or data stream) for each of the left and right eyes [201]. Rolling temporal segmentation of this data stream may then be performed [202]. The preferred size of the segments (minimum/maximum) may typically depend on a range of seizure lengths that are expected to be observed. One approach is to check for several of them at the same time, and not pre-assign a length ahead of time. In the example shown, temporal rolling segments of 1 second, 2 seconds, 5 seconds, up to N seconds are taken.

The rolling segments may then be subjected to pre-processing [203], prior to analysis for seizure detection [204]. Pre-processing may include, for example, Fast Fourier Transform (FFT), wavelet transform, or some other approach to selecting frequency or other signal components of interest. An FFT is more traditional, and usually more optimized in terms of computational efficiency. Wavelet transforms are newer, but often more suited to transient signals such as those we anticipate seeing from seizures. The pre-processing [203] may also include other types of signal processing such as low- or band-pass filtering performed prior to or in place of any FFT or wavelet operations.

In some instances, it is expected that an FFT or wavelet (or other) transform may not even be necessary. For example, the detection algorithm [204] may employ a neural-network that learns the correct weights (given enough data) directly from the time-domain. However, it is expected that such frequency domain transforms are often how humans are able to analyze (and detect) signals of interest visually, so it may be a basis to start with. In general, we expect that with limited data, more pre-processing is preferred as it can add contrast to the events of interest for detection by the algorithm [204].

In an approach as shown here, where segment sizes of different lengths are available, a “likelihood per length scale” may be determined [205]. The likelihood may be the detection confidence/probability that naturally falls out of the machine learning algorithm(s) used. For example, for each of the possible segment lengths, (1 s seizure, 2 s seizure, 5 seizure, etc.) this step may return a % detection value.

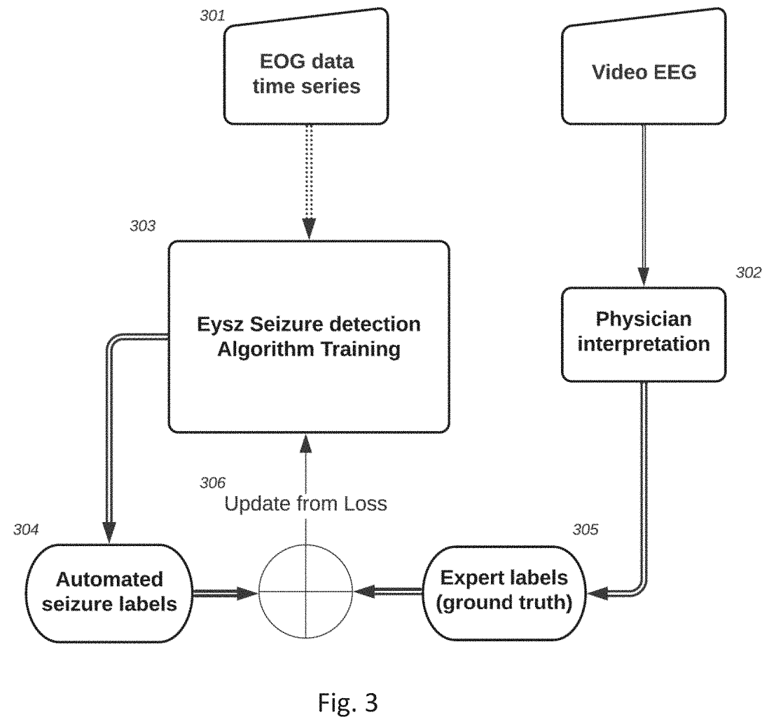

In one embodiment, the seizure detection algorithm [204] may use one or more neural networks to learn examples of EOG signals indicative of conditions of interest. Generally speaking, the neural network should be “pre-trained” using inputs that are known with a high confidence level to be indicative of seizures (or drowsiness or other conditions of interest), as described schematically in FIG. 3. Here, as part of the algorithm development, physician/doctor interpretations [302] provide labels [305] are used as ground-truth for training [303], an iterative process that uses the difference between the output [304] of the algorithm [204] and the expert labels [305] as a loss function for improvement. Depending on the neural network (or other machine learning algorithm) applied for training [303], the process may leverage both known good and known bad examples to validate a result.

The above is thus one approach to use electrooculography (EOG) signals (e.g. as output from electrodes placed on the skin near the eyes) to identify patterns associated with or predictive of seizures, syncope, drowsiness and loss of consciousness etc. It should be understood however that this invention is not limited to any particular algorithm to process or identify such patterns.

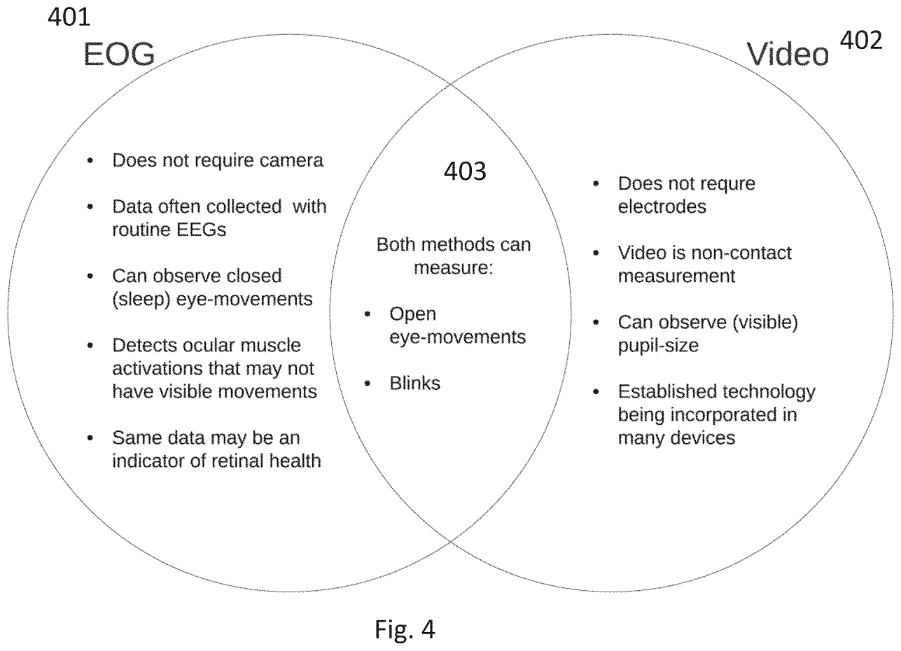

FIG. 4 is a chart comparing how EOG and video may be used by neurologists to identify seizures. EOG is typically subtracted from the EEG as an artifact and not analyzed significantly. Currently, physicians use passive observation of video to determine if a patient has lost consciousness (for example interacting with a caregiver at the time of a seizure). The only way to measure change in consciousness is through confrontational testing-asking the patient to perform a task or respond to questions. Currently, seizure is a definition requiring clear clinical correlation. Briefer bursts of epileptiform activity, or longer events which are more localized in the brain (such as those recorded with direct brain recording) can be difficult to determine if these events are clinically relevant and thus appropriate for treatment adjustment. The goal of the EOG based system described herein is to identify which electrical events are associated with clinical consciousness changes that although subtle on video are known to result in significant clinical impact including injury. Compared to the other methods, the techniques described herein have significant advantages over the video-only based methods. EOG can be included as part of an EEG producing an enriched data flow and interpretation of clinical events. EOG when used alone requires significantly less battery usage allowing for longer recording periods, as well as recording during sleep and can be integrated into eyewear with minimal footprint.

Implementation Variations

The foregoing description of example embodiments illustrates and describes systems and methods for implementing a system and/or method and/or device for using EOG to detect and characterize seizures, drowsiness, and other conditions. However, it is not intended to be exhaustive or limited to the precise form disclosed.

The embodiments described above may be implemented in many different ways. In some instances, the various “data processing systems” may each be implemented by a separate or shared physical or virtual general-purpose computer having one or more central processor(s), memor(ies), disk or other mass storage device(s), communication interface(s), input/output (I/O) device(s), and other peripherals. The general-purpose computer is transformed into a processor with improved functionality, and executes the processes described above to provide improved operations. The processors may operate, for example, by loading software instructions, and then executing the instructions to carry out the functions described.

As is known in the art, such a computer may contain a system bus, where a bus is a set of hardware wired connections used for data transfer among the components of a computer or processing system. The bus or busses are shared conduit(s) that connect different elements of the computer system (e.g., processor, disk storage, volatile and non-volatile memory, input/output ports, network ports, etc.) to enable the transfer of information. One or more central processor units are attached to the system bus and provide for the execution of computer instructions. Also attached to the system bus are typically I/O device interfaces for connecting various input and output devices (e.g., keyboard, mouse, displays, printers, speakers, etc.) to the computer. Network interface(s) allow the computer to connect to various other devices attached to a network. Memory provides volatile or non-volatile storage for computer software instructions and data used to implement an embodiment. Disk or other mass storage provides non-volatile storage for computer software instructions and data used to implement, for example, the various procedures described herein.

Embodiments may therefore typically be implemented in hardware, firmware, software, or any combination thereof. In some implementations, the computers that execute the processes described above may be deployed in a cloud computing arrangement that makes available one or more physical and/or virtual data processing machines via a convenient, on-demand network access model to a shared pool of configurable computing resources (e.g., networks, servers, storage, applications, and services) that can be rapidly provisioned and released with minimal management effort or service provider interaction. Such cloud computing deployments are relevant and typically preferred as they allow multiple users to access computing. By aggregating demand from multiple users in central locations, cloud computing environments can be built in data centers that use the best and newest technology, located in the sustainable and/or centralized locations and designed to achieve the greatest per-unit efficiency possible.

Although certain data processing systems, such as the recovery data processing systems, are described as providing a “service” to the “customers” that operate data processing systems, it should be understood that the systems may be operated as part of the same enterprise, college campus, research institution, etc., where there are no actual human or corporate “customers” that pay money to access a “service”.

Furthermore, firmware, software, routines, or instructions may be described herein as performing certain actions and/or functions. It also should be understood that the block and network diagrams may include more or fewer elements, be arranged differently, or be represented differently. Therefore, it will be appreciated that such descriptions contained herein are merely for convenience and that such actions in fact result from computing devices, processors, controllers, or other devices executing the firmware, software, routines, instructions, etc.

Other modifications and variations are possible in light of the above teachings. For example, while a series of steps has been described above with respect to the flow diagrams, the order of the steps may be modified in other implementations. In addition, the steps, operations, and steps may be performed by additional or other modules or entities, which may be combined or separated to form other modules or entities. For example, while a series of steps has been described with regard to certain figures, the order of the steps may be modified in other implementations consistent with the principles of the invention. Further, non-dependent steps may be performed in parallel. Further, disclosed implementations may not be limited to any specific combination of hardware.

Certain portions may be implemented as “logic” that performs one or more functions. This logic may include hardware, such as hardwired logic, an application-specific integrated circuit, a field programmable gate array, a microprocessor, software, firmware, or a combination thereof. Some or all of the logic may be stored in one or more tangible non-transitory computer-readable storage media and may include computer-executable instructions that may be executed by a computer or data processing system. The computer-executable instructions may include instructions that implement one or more embodiments described herein. The tangible non-transitory computer-readable storage media may be volatile or non-volatile and may include, for example, flash memories, dynamic memories, removable disks, and non-removable disks.

Accordingly, further embodiments may also be implemented in a variety of computer architectures, physical, virtual, cloud computers, and/or some combination thereof, and thus the computer systems described herein are intended for purposes of illustration only and not as a limitation of the embodiments.

In practicing the subject methods, determining the presence or absence of a change in an EOG signal may involve machine learning. Machine learning techniques and computational methods may be used for predicting seizures, syncope, drowsiness, loss of consciousness or other neurological events or conditions from the data obtained. The machine learning process may involve relating the numerical data to the outcomes, which applies categorical training to detect and/or predict a condition or event.

In certain aspects, machine learning models may include aspects of signal acquisition, signal preprocessing, features extraction from the signals, and classification between different seizure states. The disclosed methods and systems may also include confirming the presence or absence of a change relative to baseline, perform lower order statistical analysis and/or a higher order statistical analysis of the data. In other embodiments, the condition or event in the subject is detected and/or predicted in the absence of measuring an EOG signal of the subject.

Open source tools may be employed to develop the methods described herein. This may include numerical processing languages such as Python or R, and deep learning development toolkits, such as TensorFlow, PyTorch, and Keras to name a few.

Commercially available tools such as MATLAB's Statistics and Machine Learning Toolbox™, Neural Network Toolbox™, Image Processing Toolbox™, the Image Acquisition Toolbox™, Mapping Toolbox™ and other MATLAB tools, such as the MATLAB Signal Processing Toolbox™ may also be leveraged to provide the machine learning and signal processing methods described herein.

The EOG data in a time series may be also analyzed by a lower order statistical analysis and/or a higher order statistical analysis including, but not limited to, mean, standard deviation, kurtosis, and dominant frequencies from spectral analysis of the EOG data. For example, a sequence of learning procedures listed by increasing processing complexity may be numerical data obtained from a EOG measuring device analyzed using a lower order statistical analysis and/or a higher order statistical analysis, categorical outcomes produced by a clinical read, and lastly, associating the numerical data to the categorical data. In certain aspects, the disclosed methods herein utilize machine learning algorithms embedded in-line with the disclosed methods to enhance clinical practices in identifying subjects as having an event or condition.

In some embodiments, machine learning algorithms involve thresholding as determined by a statistical reliability of outcomes. In some embodiments, a portion of the data obtained may be used for training and the remaining data for testing and determining statistical analysis of outcomes. In such cases, the data breakdown is analogous to a standard 2×2 decision theory representation of true/false positives and true/false negatives. For example, a receiver operating characteristic curve (ROC curve) may be created to illustrate the true positive rate against the false positive rate at various threshold settings. The true-positive rate is also known as sensitivity, recall or probability of detection in machine learning.

No element, act, or instruction used herein should be construed as critical or essential to the disclosure unless explicitly described as such. Also, as used herein, the article “a” is intended to include one or more items. Where only one item is intended, the term “one” or similar language is used. Further, the phrase “based on” is intended to mean “based, at least in part, on” unless explicitly stated otherwise.

Headings and/or subheadings herein are used to segment this patent application into portions to facilitate the readability of the application. These headings and/or subheadings are not intended to define or limit the scope of what is disclosed and/or claimed in this patent application.

Also, the term “user”, as used herein, is intended to be broadly interpreted to include, for example, a computer or data processing system or a human user of a computer or data processing system, unless otherwise stated.

The above description contains several example embodiments. It should be understood that while a particular feature may have been disclosed above with respect to only one of several embodiments, that particular feature may be combined with one or more other features of the other embodiments as may be desired and advantageous for any given or particular application. It is, of course, not possible to describe every conceivable combination of components or methodologies for purposes of describing the innovations herein, and one skill in the art may now, in light of the above description, recognize that many further combinations and permutations are possible. Also, to the extent that the terms “includes,” and “including” and variants thereof are used in either the detailed description or the claims, these terms are intended to be inclusive in a manner similar to the term “comprising”.

The above description contains several example embodiments. It should be understood that while a particular feature may have been disclosed above with respect to only one of several embodiments, that particular feature may be combined with one or more other features of the other embodiments as may be desired and advantageous for any given or particular application. It is, of course, not possible to describe every conceivable combination of components or methodologies for purposes of describing the innovations herein, and one skill in the art may now, in light of the above description, recognize that many further combinations and permutations are possible.

Accordingly, the claimed subject matter is intended to embrace all such alterations, modifications, and variations that fall within the spirit and scope of the appended claims. Furthermore, to the extent that the terms “includes,” and “including” and variants thereof are used in either the detailed description or the claims, these terms are intended to be inclusive in a manner similar to the term “comprising”.

1. A method comprising:
detecting one or more electrooculographic (EOG) signals for each of a left eye and/or right eye of a subject;
converting the one or more EOG signals to EOG time series data;
storing the EOG time series data; and
analyzing the stored EOG time series data to provide an output indicative and/or predictive of seizures, syncope, drowsiness, loss of consciousness, or other neurological events or conditions of the subject.

2. The method of claim 1 wherein the EOG signals are first collected from a source that produces combined electroencephalographic (EEG) and EOG signals, additionally comprising:
before the step of detecting, separating the EOG signals indicative of extraocular muscle activation or dipole movement from the EEG signals.

3. The method of claim 1 wherein the analyzing step further comprises:
combining the EOG time series data with video data or data derived from other eye-movement detection devices.

4. The method of claim 1 wherein the EOG time series data is derived from a device that produces other data such as EEG data.

5. The method of claim 1 additionally comprising:
converting the EOG time series data into relative eye-movement vectors; and
further analyzing the relative eye-movement vectors to determine resulting changes in eye-movement that are patterns associated with the seizures, syncope, drowsiness, loss of consciousness, or other neurological events or conditions.

6. The method of claim 2 additionally comprising:
temporally segmenting the EOG time series data to provide temporally segmented EOG data;
preprocessing the temporally segmented EOG data with one or more signal processing steps.

7. The method of claim 1 additionally comprising:
producing structured information as a result of one of analyzing steps; and
transmitting the structured information electronically to another computing device for further action by another system or human to contribute to a medical or scientific decision.

8. The method of claim 1 wherein the condition is a seizure, and additionally comprising:
producing an alarm or communicating with a neurostimulation or other closed-loop device, such as a Vagus Nerve Stimulator (VNS) or Responsive Neurostimulation System (RNS), to stop the seizure.

9. The method of claim 1 additionally comprising:
generating a training data set, the training data set including labels known to be indicative of seizures, seizures, syncope, drowsiness, loss of consciousness, or other neurological events or conditions of the subject; and
validating the output of the analyzing step by further matching against the training data set.

10. The method of claim 9 wherein the validating step further comprises one or more of a neural network or other machine learning algorithm.

11. A method for controlling seizures of a human subject using electrooculographic (EOG) signals comprising:
collecting electroencephalographic (EEG) signals from the human subject;
separating EOG signals indicative of ocular muscle activation for each of a left eye and right eye from the EEG signals;
converting the EOG signals into EOG time series data;
storing the EOG time series data;
temporally segmenting the EOG time series data to provide temporally segmented EOG data;
pre-processing the temporally segmented EOG data with one or more signal processing steps to provide preprocessed EOG data;
obtaining a training data set including labels for EOG data known to be indicative of seizures;
training a neural network using the training data set and the temporally segmented EOG data;
matching the preprocessed EOG data against the neural network; and
further controlling a seizure in the human subject by one or more of
producing an alarm;
further communicating with a neurostimulation or other closed-loop device—such as a Vagus Nerve Stimulator (VNS) or Responsive Neurostimulation System (RNS).
